(19) United States
(12) Patent Application Publication
(10) Pub. No.: US 2011/0107821 A1
(43) Pub. Date: May 12, 2011

(54) MULTIMARKER PANEL FOR MONITORING PATIENTS WITH AND WITHOUT OVERT HEART FAILURE

(76) Inventors: Georg Hess, Mainz (DE); Andrea Horsch, Mannheim (DE); Dietmar Zdunek, Tutzing (DE)

(21) Appl. No.: 13/005,805

(22) Filed: Jan. 13, 2011

Related U.S. Application Data
(63) Continuation of application No. PCT/EP2009/058948, filed on Jul. 14, 2009.

(30) Foreign Application Priority Data
Jul. 14, 2008 (EP) 08160319.3

(51) Int. Cl. G01N 33/48

(52) U.S. Cl. 73/61.43

(57) ABSTRACT

The present invention relates to a method for monitoring a subject suffering from heart failure, the method involving repeatedly determining, within given time intervals, the amounts of each of the following peptide markers: NT-proANP or a variant thereof; NT-proBNP or a variant thereof; a cardiac troponin or a variant thereof; and GDF-15 or a variant thereof; in a sample from the subject; and comparing the amounts measured in each determination with reference amounts of each marker; and assessing, based on differences in determined amounts in one or more of the markers, whether the subject is stable or has undergone a change in pathophysiological state.

### RELATED APPLICATIONS

This application is a continuation of PCT/EP2009/058948 filed Jul. 14, 2009 and claims priority to EP 08160319.3 filed Jul. 14, 2008.

### FIELD OF THE INVENTION

The present invention relates to diagnostic means and methods. Specifically, the present invention relates to a method for monitoring patients with overt heart failure by measuring the amounts of NT-proBNP, NT-proANP, a cardiac troponin and GDF-15 and establishing a differential diagnosis of the variations of the pathophysiological state of the patient. Further, it relates to a method of diagnosing to which therapy a subject suffering from heart failure is susceptible. Finally, the present invention encompasses diagnostic devices and kits for carrying out the aforementioned methods.

### BACKGROUND OF THE INVENTION

B-type natriuretic peptides (BNP and NT-proBNP) are well-known and established markers for the identification of persons with cardial dysfunction and heart failure. In particular, many studies have shown that BNP and NT-proBNP are predictive for the outcome of various persons and patient groups (Gustafsson et al., J. Card. Fail. 2005, 11, S. 15-S. 20; Gardner et al., Eur. Heart J. 2003, 24, p. 1735-1743). This holds in particular true for patients in various stages of heart failure (also named cardiac insufficiency) and persons suffering from acute coronary syndrome (“ACS”). A change in the cardiac function induces a change in the concentration of the natriuretic peptides. These changes may be caused by the therapy or by a change in the underlying disease. In consequence, natriuretic peptides have been recommended for therapy monitoring and/or as therapeutic targets.

The levels of the natriuretic peptides can change even in stable heart failure patients, and the causes of this phenomenon are still unknown. It was found that the change occurs slowly. Within a time period of 1 week, changes in the natriuretic peptide levels which are not understood (i.e. without any evident change in the pathophysiological status of the patient) are rare. In longer time periods (e.g. 2 years), the levels may change up to 100% (Schou et al., European Heart Journal 2007, 28, p. 177-182; Schou et al., European Journal of Heart Failure 2007, 9, p. 68-74). It is furthermore known that the level of B-type natriuretic peptides may vary as a consequence of a change in renal function, water balance or cardiac rhythm (Goei et al., Am. J. Cardiol. 2008, 1, p. 122-126; EP-A 1 577 673).

Accordingly, measuring the level on a B-type natriuretic peptide or NT-proBNP alone does not provide sufficient information allowing to assess if patients suffering from heart failure which are regarded as being stable or unstable in accordance with clinical signs are in fact stable/unstable and/or are suffering from complications affecting their cardiac state. It would be desirable to have a method which would allow to establish if an individual is undergoing an amelioration or a deterioration in their pathophysiological state or if this state is stable. Furthermore, it would be desirable to have a method which would allow to monitor if the pathophysiological state of an individual deteriorates or ameliorates, for example after an intervention. In particular, the individual is an individual suffering from heart failure.

Thus, the technical problem underlying the present invention is to be seen as the provision of methods and means for complying with the aforementioned needs. The technical problem is solved by the embodiments characterized in the accompanying claims and herein below.

### SUMMARY OF THE INVENTION

Accordingly, the present invention relates to a method for monitoring a subject suffering from heart failure, the method comprising:
a) repeatedly determining, within given time intervals, the amounts of each of the following peptides:
NT-proANP or a variant thereof; NT-proBNP or a variant thereof;
a cardiac troponin or a variant thereof;
GDF-15 or a variant thereof;
in a sample of the said subject; and
b) comparing the amounts measured in each determination of each of the markers cited in step a) and comparing these with reference amounts; and
c) assessing, based on the differences in the determined amounts in one or more of the above-cited markers, whether the subject is stable or has undergone a change in its pathophysiological state

In other words, the method according to the present invention allows to determine whether a subject suffering from heart failure undergoes or has undergone a change in its pathophysiological state, without any apparent changes in its pathophysiological state being or having been noted.

The subject is a stable or unstable subject, or even an apparently stable or apparently unstable subject.

### BRIEF DESCRIPTION OF THE DRAWINGS

FIG. 1 depicts the variation in the markers in the study described in Example 1, illustrating the 5th, 25th, 75th and 95th percentile.

FIGS. 2 and 3 show the variation in the levels of the peptides studied in Example 2, depending on the time interval and the pathophysiological state of the patient. FIG. 2 shows 29 patients returned to cardiac sinus rhythm, and FIG. 3 shows 10 patients who continued with cardiac fibrillation.

### DETAILED DESCRIPTION OF THE INVENTION

In the present context, “stable” refers to the clinical state of the patient and is indicative for the fact that no subjective changes in the medical history (i.e. change of symptoms and of clinical evidence) of the patient occur, in connection with a stability in the patient's weight and an unchanged medication. This state is also referred to as “apparently stable”.

In the context of the present invention, “apparently stable” refers to the situation that the person skilled in the art (in general a physician) will consider that the patient does not undergo any changes in his pathophysiological state, due to information gathered from usual inspection/examination of the patient. Preferably, this inspection/examination will not go further than routine examination, i.e. including visual inspection, measuring of blood pressure, and imaging.

As is clear to the person skilled in the art, the inverse of the above holds true for unstable subjects, i.e. the subject is undergoing subjective changes, and the person skilled in the art will consider the pathophysiological state to be unstable.

More preferably, the examination does not include invasive methods. In particular are appropriate chest x-ray, stress testing, cardiac ventriculography, ECG, echocardiography, coronary angiography, X-ray. In particular, the examination does not include the determination of molecular markers characteristic for physiological changes in the myocard. These markers are, in principle, known to the person skilled in the art and include (besides the markers or peptides used in the context of the present invention) myoglobin, D-dimer, CK-MB, endoglin, VEGF, PlGF, sFLT1, CD40, CD40L, sCD40L, H-FABP (fatty acid binding protein), MPO, creatinin, electrolytes, blood count.

The term “pathophysiological state” in the context of the present invention in particular refers to the myocard. Accordingly, any improvement or deterioration in the state of the myocard (e.g. cell death, wall stress, inflammatory processes, atherosclerosis of the myocard vessels, thrombosis) is a change in the pathophysiological state.

The method of the present invention may be also used for diagnosing, confirmation, and subclassification of the pathophysiological state of a subject. The method may be carried out manually or assisted by automation. Preferably, step (a) and/or (b) may in total or in part be assisted by automation, e.g., by a suitable robotic and sensory equipment for the determination in step (a) or a computer-implemented comparison in step (b).

The term “monitoring” as used herein refers to supervising or controlling the pathophysiological state of a subject. In other words, the method of the present invention is keeping track or getting aware of any changes in the pathophysiological state of a subject. Preferably, the method is applied for monitoring patients in which a change in their pathophysiological state is not evident (i.e. the patient does not show obvious signs of a change in the pathophysiological state, or the patient is suspicious of having undergone a change in its pathophysiological state, but the state can be monitored/determined only with elaborate, time-consuming and costly methods, in particular methods other than determining the level of molecular “markers”, in general polypeptides which are expressed in response to the occurrence of a certain physiological phenomenon). As will be understood by those skilled in the art, such an assessment is usually not intended to be correct for 100% of the subjects to be diagnosed. The term, however, requires that a statistically significant portion of subjects can be correctly diagnosed to exhibit the change in the pathophysiological state. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the diagnosis will be correct for at least 60%, at least 70%, at least 80%, or at least 90% of the subjects of a given cohort or population.

The term “repeatedly determining the amounts”, in the context of the present invention, means that the amounts of the respective peptide in a body liquid of the individual are determined at least twice in the course of the monitoring process of the present invention. In general, the first measurement will take place at the starting point of the monitoring of the individual (i.e. the monitoring process starts with the first determination of the amounts of the respective peptides). The end point of the monitoring may be reached when the amount of at least one of the determined peptides shows that the individual is not stable. The end point may also be reached when the individual has been diagnosed as being healthy or stable, either by the method of the present invention or by other diagnostic methods known to the person skilled in the art.

Between the starting point and the end point of the method according to the invention, a various number of determinations of the amount of the respective peptide(s) can be carried out. An extreme case is that the first measurement is the starting point and the second measurement is the end point. Of course, there can be carried out 2, 3, 4, 5, 6, 7, 8, 9, 10, 11, 12, 13, 14, 15, or even more determinations of the amounts of each of the peptides relevant in the context of the present invention.

The time intervals between the individual determinations of the respective peptides may vary within various limits. The time interval may be 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 1 month, 2 months, 3 months, 4 months, 5 months, 6 months or even longer.

The method of the present invention, preferably, is an in vitro method. Moreover, it may comprise steps in addition to those explicitly mentioned above. For example, further steps may relate to sample pre-treatments or evaluation of the results obtained by the method.

The term “subject” as used herein relates to animals, preferably mammals, and, more preferably, humans. However, it is envisaged by the present invention that the subject shall be suffering from heart failure.

The term “heart failure” as used herein relates to an impaired systolic and/or diastolic function of the heart. Preferably, heart failure referred to herein is also chronic heart failure. Heart failure can be classified into a functional classification system according to the New York Heart Association (NYHA). Patients of NYHA Class I have no obvious symptoms of cardiovascular disease but already have objective evidence of functional impairment. Physical activity is not limited, and ordinary physical activity does not cause undue fatigue, palpitation, or dyspnea (shortness of breath). Patients of NYHA class II have slight limitation of physical activity. They are comfortable at rest, but ordinary physical activity results in fatigue, palpitation, or dyspnea. Patients of NYHA class III show a marked limitation of physical activity. They are comfortable at rest, but less than ordinary activity causes fatigue, palpitation, or dyspnea. Patients of NYHA class IV are unable to carry out any physical activity without discomfort. They show symptoms of cardiac insufficiency at rest. Heart failure, i.e., an impaired systolic and/or diastolic function of the heart, can be determined also by, for example, echocardiography, angiography, scintigraphy, or magnetic resonance imaging. This functional impairment can be accompanied by symptoms of heart failure as outlined above (NYHA class II-IV), although some patients may present without significant symptoms (NYHA I). Moreover, heart failure is also apparent by a reduced left ventricular ejection fraction (LVEF). More preferably, heart failure as used herein is accompanied by a left ventricular ejection fraction (LVEF) of less than 60%, of 40% to 60% or of less than 40%.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

The terms peptide, polypeptide, and protein are used interchangeably throughout this specification.

The term “natriuretic peptide” comprises Atrial Natriuretic Peptide (ANP)-type and Brain Natriuretic Peptide (BNP)-type peptides and variants thereof having the same predictive potential. Natriuretic peptides according to the present invention comprise ANP-type and BNP-type peptides and variants thereof (see e.g. Bonow, 1996, Circulation 93: 1946-1950). ANP-type peptides comprise pre-proANP, proANP, NT-proANP, and ANP. BNP-type peptides comprise pre-proBNP, proBNP, NT-proBNP, and BNP. The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP).

ANP-type and BNP-type peptides belong to the group of natriuretic peptides (see e.g. Bonow, R. O. (1996). New insights into the cardiac natriuretic peptides. Circulation 93: 1946-1950).

ANP-type peptides comprise pre-proANP, proANP, NT-proANP, and ANP.

BNP-type peptides comprise pre-proBNP, proBNP, NT-proBNP, and BNP.

The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP).

Preferred natriuretic peptides according to the present invention are NT-proANP, ANP, NT-proBNP, BNP, and variants thereof. ANP and BNP are the active hormones and have a shorter half-life than their respective inactive counterparts, NT-proANP and NT-proBNP. BNP is metabolized in the blood, whereas NT-proBNP circulates in the blood as an intact molecule and as such is eliminated renally. The in-vivo half-life of NT-proBNP is 120 min longer than that of BNP, which is 20 min (Smith M W, Espiner E A, Yandle T G, Charles C J, Richards A M. Delayed metabolism of human brain natriuretic peptide reflects resistance to neutral endopeptidase. J. Endocrinol. 2000; 167: 239-46.).

BNP is produced predominantly (albeit not exclusively) in the ventricle and is released upon increase of wall tension. Thus, an increase of released BNP reflects predominantly dysfunctions of the ventricle or dysfunctions which originate in the atria but affect the ventricle, e.g. through impaired inflow or blood volume overload.

In contrast, ANP is produced and released exclusively from the atrium. The level of ANP may therefore predominantly reflect atrial function.

Preanalytics are robust with NT-proBNP, which allows easy transportation of the sample to a central laboratory (Mueller T, Gegenhuber A, Dieplinger B, Poelz W, Haltmayer M. Long-term stability of endogenous B-type natriuretic peptide (BNP) and amino terminal proBNP (NT-proBNP) in frozen plasma samples. Clin Chem Lab Med 2004; 42: 942-4.). Blood samples can be stored at room temperature for several days or may be mailed or shipped without recovery loss. In contrast, storage of BNP for 48 hours at room temperature or at 4° Celsius leads to a concentration loss of at least 20% (Mueller T, Gegenhuber A, et al., Clin Chem Lab Med 2004; 42: 942-4, supra; Wu A H, Packer M, Smith A, Bijou R, Fink D, Mair J, Wallentin L, Johnston N, Feldcamp C S, Haverstick D M, Ahnadi C E, Grant A, Despres N, Bluestein B, Ghani F. Analytical and clinical evaluation of the Bayer ADVIA Centaur automated B-type natriuretic peptide assay in patients with heart failure: a multisite study. Clin Chem 2004; 50: 867-73.).

Therefore, depending on the time-course or properties of interest, either measurement of the active or the inactive forms of the natriuretic peptide can be advantageous.

The term “variants” in this context relates to peptides substantially similar to said peptides. The term “substantially similar” is well understood by the person skilled in the art. In particular, a variant may be an isoform or allele which shows amino acid exchanges compared to the amino acid sequence of the most prevalent peptide isoform in the human population. Preferably, such a substantially similar peptide has a sequence similarity to the most prevalent isoform of the peptide of at least 80%, preferably at least 85%, more preferably at least 90%, most preferably at least 95%. Substantially similar are also proteolytic degradation products which are still recognized by the diagnostic means or by ligands directed against the respective full-length peptide.

The term “variant” also relates to a post-translationally modified peptide such as glycosylated peptide. A “variant” is also a peptide which has been modified after collection of the sample, for example by covalent or non-covalent attachment of a label, particularly a radioactive or fluorescent label, to the peptide. Measuring the level of a peptide modified after collection of the sample is understood as measuring the level of the originally non-modified peptide.

In the context of the present invention, the most preferred natriuretic peptides are, on the one hand, NT-proBNP and, on the other hand, NT-proANP.

The term “cardiac Troponin” refers to all Troponin isoforms expressed in cells of the heart and, preferably, the subendocardial cells. These isoforms are well characterized in the art as described, e.g., in Anderson 1995, Circulation Research, vol. 76, no. 4: 681-686 and Ferrieres 1998, Clinical Chemistry, 44: 487-493. Preferably, cardiac Troponin refers to Troponin T and/or Troponin I, and, most preferably, to Troponin T. It is to be understood that isoforms of Troponins may be determined in the method of the present invention together, i.e. simultaneously or sequentially, or individually, i.e. without determining the other isoform at all. Amino acid sequences for human Troponin T and human Troponin I are disclosed in Anderson, loc cit and Ferrieres 1998, Clinical Chemistry, 44: 487-493.

The term “cardiac Troponin” encompasses also variants of the aforementioned specific Troponins, i.e., preferably, of Troponin I, and more preferably, of Troponin T. Such variants have at least the same essential biological and immunological properties as the specific cardiac Troponins. In particular, they share the same essential biological and immunological properties if they are detectable by the same specific assays referred to in this specification, e.g., by ELISA Assays using polyclonal or monoclonal antibodies specifically recognizing the said cardiac Troponins. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific Troponin. Variants may be allelic variants or any other species specific homologs, paralogs, or orthologs. Moreover, the variants referred to herein include fragments of the specific cardiac Troponins or the aforementioned types of variants as long as these fragments have the essential immunological and biological properties as referred to above. Such fragments may be, e.g., degradation products of the Troponins. Further included are variants which differ due to posttranslational modifications such as phosphorylation or myristylation.

A particularly preferred Troponin T assay in the context of the present invention is the Elecsys® 2010 analyzer (Roche Diagnostics) with a detection limit of from 0.001 ng/ml to 0.0015 ng/ml.

The term “Growth-Differentiation Factor-15” or “GDF-15” relates to a polypeptide being a member of the transforming growth factor (TGF)-β cytokine superfamily. GDF-15 was originally cloned as macrophage-inhibitory cytokine-1 and later also identified as placental transforming growth factor-β, placental bone morphogenetic protein, non-steroidal anti-inflammatory drug-activated gene-1, and prostate-derived factor (Bootcov loc cit; Hromas, 1997 Biochim Biophys Acta 1354:40-44; Lawton 1997, Gene 203:17-26; Yokoyama-Kobayashi 1997, J Biochem (Tokyo), 122:622-626; Paralkar 1998, J Biol Chem 273:13760-13767). Similar to other TGF-β-related cytokines, GDF-15 is synthesized as an inactive precursor protein, which undergoes disulfide-linked homodimerization. Upon proteolytic cleavage of the N-terminal pro-peptide, GDF-15 is secreted as a ˜28 kDa dimeric protein (Bauskin 2000, Embo J19:2212-2220). Amino acid sequences for GDF-15 are disclosed in WO99/06445, WO00/70051, WO2005/113585, Bottner 1999, Gene 237: 105-111, Bootcov loc. cit, Tan loc. cit., Baek 2001, Mol Pharmacol 59: 901-908, Hromas loc cit, Paralkar loc cit, Morrish 1996, Placenta 17:431-441 or Yokoyama-Kobayashi loc cit. GDF-15 as used herein encompasses also variants of the aforementioned specific GDF-15 polypeptides. Such variants have at least the same essential biological and immunological properties as the specific GDF-15 polypeptides. In particular, they share the same essential biological and immunological properties if they are detectable by the same specific assays referred to in this specification, e.g., by ELISA assays using polyclonal or monoclonal antibodies specifically recognizing the said GDF-15 polypeptides. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific GDF-15 polypeptides. Moreover, the variants referred to herein include fragments of the specific GDF-15 polypeptides or the aforementioned types of variants as long as these fragments have the essential immunological and biological properties as referred to above. Such fragments may be, e.g., degradation products of the GDF-15 polypeptides. Further included are variants which differ due to posttranslational modifications such as phosphorylation or myristylation. A preferred GDF-15 assay in the context of the present invention is the assay as described by Wollert et al. in Clinical Chemistry 53, No 2, 2007, p. 284-291.

Determining the amount of the peptides or polypeptides referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

Preferably, determining the amount of a peptide or polypeptide comprises the steps of (a) contacting a cell capable of eliciting a cellular response, the intensity of which is indicative of the amount of the peptide or polypeptide with the said peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g. a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at a mass to charge (m/z) variable specific for the peptide or polypeptide observed in mass spectra or a NMR spectrum specific for the peptide or polypeptide.

Determining the amount of a peptide or polypeptide may, preferably, comprise the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g. nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display. Antibodies as referred to herein include both polyclonal and monoclonal antibodies, as well as fragments thereof, such as Fv, Fab and F(ab)2 fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g. according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g. by NMR or surface plasmon resonance.

Second, if the ligand also serves as a substrate of an enzymatic activity of the peptide or polypeptide of interest, an enzymatic reaction product may be measured (e.g. the amount of a protease can be measured by measuring the amount of cleaved substrate, e.g. on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable label prior to the reaction. Preferably, the sample is contacted with the substrate for an adequate period of time. An adequate period of time refers to the time necessary for a detectable, preferably measurable, amount of product to be produced. Instead of measuring the amount of product, the time necessary for appearance of a given (e.g. detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-galactosidase, luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF (Amersham Biosciences). A suitable enzyme-substrate combination may result in a colored reaction product, fluorescence or chemoluminescence, which can be measured according to methods known in the art (e.g. using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, fluorescein, and the Alexa dyes (e.g. Alexa 568). Further fluorescent labels are available e.g. from Molecular Probes (Oregon). Also the use of quantum dots as fluorescent labels is contemplated. Typical radioactive labels include 35S, 125I, 32P, 33P and the like. A radioactive label can be detected by any method known and appropriate, e.g. a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamid gel electrophoresis (SDS-PAGE), Western Blotting, and mass spectrometry), can be used alone or in combination with labeling or other detection methods as described above.

The amount of a peptide or polypeptide may be, also preferably, determined as follows: (a) contacting a solid support comprising a ligand for the peptide or polypeptide as specified above with a sample comprising the peptide or polypeptide and (b) measuring the amount peptide or polypeptide which is bound to the support. The ligand, preferably chosen from the group consisting of nucleic acids, peptides, polypeptides, antibodies and aptamers, is preferably present on a solid support in immobilized form.

Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20(1):9-12). In such suspension arrays, the carrier, e.g. a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide or peptide, the relative amount or concentration of the said polypeptide or peptide as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison referred to in the method of the present invention may be carried out manually or computer-assisted. For a computer-assisted comparison, the value of the determined amount may be compared to values corresponding to suitable references which are stored in a database by a computer program. The computer program may further evaluate the result of the comparison, i.e. automatically provide the desired assessment in a suitable output format.

Based on the comparison of the amount determined, and the reference amount, it is possible to identify individuals which are stable or which undergo a deterioration/amelioration of their pathophysiological state. Therefore, the reference amount is to be chosen so that either a difference or a similarity in the compared amounts allows identifying those apparently stable or apparently unstable subjects which have heart failure.

In one embodiment, the reference amounts, preferably, define thresholds. Suitable reference amounts or threshold amounts may be determined by the method of the present invention from a reference sample to be analyzed together, i.e. simultaneously or subsequently, with the test sample. A reference amount serving as a threshold may be derived from the upper limit of normal (ULN), i.e. the upper limit of the physiological amount to be found in a population of subjects (e.g. patients enrolled for a clinical trial). The ULN for a given population of subjects can be determined by various well known techniques. A suitable technique may be to determine the median of the population for the peptide or polypeptide amounts to be determined in the method of the present invention.

Accordingly, the term “reference amounts” as used herein refers to amounts of the polypeptides which allow assessing if the respective individual is stable or is undergoing/has undergone a change in his pathophysiological state.

In one aspect of the present invention, the reference amount may either be derived from (i) a subject known to be healthy or (ii) a subject known to suffer from the disease or the diseases which can be diagnosed with the specific marker. These diseases are specified hereinafter. This aspect of the invention holds true for those peptides which do not indicate heart failure, as the present invention is preferably drawn to a process for monitoring heart failure patients which accordingly show an elevated level of a heart failure indicating peptide. The other markers employed in accordance with the present invention and not indicating heart failure are indicators of pathophysiological states in the individual other than heart failure which may or may not be present at all and may develop in the monitoring process. This aspect applies in particular for cardiac troponins and GDF-15. These reference amounts thus are those of “normal” individuals in respect to the named peptides, i.e. individuals not suffering from a disease for which the release of the peptides used in the present invention, in particular GDF-15 and/or a cardiac troponin, is characteristic. A deviation from these values is characteristic for a pathophysiological state. Hereinafter, this type of reference amounts may be referred to as “healthy reference amounts”. This type of reference amounts is, in general, identical with the ULN.

In accordance with the foregoing, in a further aspect of the present invention the reference amount for the markers indicating heart failure is developed from the individual who is known to suffer from heart failure himself. These “reference values” are therefore characteristic for an existing pathophysiological state. A deviation from this value will be indicative for a change deterioration or an amelioration of the state. This aspect in particular applies for NT-proBNP and NT-proANP. Hereinafter, this type of reference amounts may be referred to as “pathological reference amounts”.

Of course, and as is clear from the foregoing, an individual suffering not only from heart failure, but also from other diseases which may be indicated by one or more of the markers used in accordance with the present invention, may have elevated amounts of this or these marker(s) right from the beginning of the monitoring process of the present invention. In these cases, the reference amount may also be developed from the individual himself, in still a further aspect of the present invention. Accordingly, in this aspect of the present invention, the levels of GDF-15 and/or a cardiac troponin, in particular troponin T, are elevated when the monitoring process starts, due to a specific pathophysiological state indicated by the named peptide/peptides. Any deviation from these reference amounts will indicate a change in the pathophysiological state.

Myocardial dysfunction is a general term, describing several pathophysiological states of the heart muscle (myocard). A myocardial dysfunction may be a temporary pathophysiological state (caused by e.g. ischemia, toxic substances, alcohol, . . . ), contrary to heart failure. Myocardial dysfunction may disappear after removing the underlying cause. A symptomless myocardial dysfunction may, however, also develop into heart failure (which has to be treated in a therapy). A myocardial dysfunction may, however, also be a heart failure, a chronic heart failure, even a severe chronic heart failure. In general, a myocardial dysfunction is an impaired systolic and/or diastolic function of the heart, and a myocardial dysfunction may occur with or without heart failure. Any heart failure mentioned beforehand my be symptomless.

The present invention therefore also relates to cardiac disorders, preferably from the group myocardial dysfunction and heart failure.

Heart failure is a condition that can result from any structural or functional cardiac disorder that impairs the ability of the heart to fill with or pump a sufficient amount of blood throughout the body. Even with the best therapy, heart failure is associated with an annual mortality of about 10%. Heart failure is a chronic disease; it can, inter alia, occur either following an acute cardiovascular event (like myocardial infarction), or it can occur e.g. as a consequence of inflammatory or degenerative changes in myocardial tissue. Heart failure patients are classified according to the NYHA system in classes I, II, III and IV. A patient having heart failure will not be able to fully restore his health without receiving a therapeutical treatment.

Myocardial dysfunction and heart failure often remain undiagnosed, particularly when the condition is considered “mild.” The conventional diagnostic techniques for heart failure are based on the well known vascular volume stress marker NT-proBNP. However, the diagnosis of heart failure under some medical circumstances based on NT-proBNP appears to be incorrect for a significant number of patients but not all (e.g., Beck 2004, Canadian Journal of Cardiology 20: 1245-1248; Tsuchida 2004, Journal of Cardiology, 44:1-11). However, especially patients which suffer from heart failure would urgently need a supportive therapy of heart failure. On the other hand, as a consequence of an incorrect diagnosis of heart failure, many patients will receive a treatment regimen which is insufficient or which may have even adverse side effects.

The following (pathological) reference values of a natriuretic peptide are considered indicative for the presence of myocardial dysfunction, in particular heart failure:

NT-proANP referred to herein: preferably 800 pg/ml, more preferably 1800 pg/ml, in particular 3000 pg/ml.

NT-proBNP referred to herein: preferably 125 pg/ml, in particular 200 pg/ml, most preferably 350 pg/ml.

Furthermore, in accordance with a preferred embodiment of the present invention, and with respect to the above-cited reference values, an increased amount of natriuretic peptide, in particular NT-proBNP and/or NT-proANP is indicative for myocardial dysfunction, in particular heart failure, whereas with respect to the reference values, a decreased amount of natriuretic peptide, in particular NT-proBNP and/or NT-proANP is indicative for the absence of myocardial dysfunction, in particular for the absence of heart failure. Thus, in a preferred embodiment of the method of the present invention, an increased amount of natriuretic peptide, in particular NT-proBNP and/or NT-proANP is indicative for myocardial dysfunction, in particular heart failure. In another preferred embodiment of the method of the present invention, a decreased amount of natriuretic peptide, in particular NT-proBNP and/or NT-proANP is indicative for the absence of myocardial dysfunction, in particular for the absence of heart failure.

Accordingly, determining the amount of a natriuretic peptide furthermore permits to assess whether the individual suffering from symptoms of heart failure is also suffering from myocardial dysfunction and/or heart failure, and if the dysfunction/failure is severe or less severe.

In advantageous embodiments of the method of the present invention, there can be obtained information from the combination of the amounts of NT-proBNP and of NT-proANP measured in a sample of an individual.

For example, in a patient presenting with symptoms of acute cardiac decompensation, an increased level of NT-proANP in presence of a non-increased or weakly increased level of NT-proBNP indicates the presence of an acute cardiac event, and/or an increased level of NT-proANP in presence of a highly increased level of NT-proBNP indicates the presence of a chronic cardiac disease.

This embodiment preferably deals with three groups of patients showing symptoms of acute cardiac decompensation: Patients suffering from acute cardiac events (1), patients suffering from chronic cardiac diseases who presently suffer from an additional acute cardiac event (2), and patients suffering from a chronic cardiac disease which is acutely decompensating (3).

Patients presenting with symptoms of acute decompensation caused by an acute cardiac event (above-mentioned patient group 1) show a non-increased or only weakly increased level of NT-proBNP but a highly increased level of NT-proANP at the time of admission to a hospital. Furthermore, in these patients the level of NT-proANP drops off rapidly within the first 12 hours after admission.

In the context of the present invention, the term “acute cardiac event” refers to events in which a deterioration of the cardiac state of the patient occurs within short time (i.e. seconds, minutes, hours or maximum 1 day). This can be shown by pain symptoms (like e.g. in ACS), dyspnea, or a collapse. The “acute” state can remain, improve or deteriorate. Causes are ischemic events, dysrythmia accompanied by hemodynamic changes, endogenous volume overload (e.g. in pump failure after myocardial infraction), exogenous volume overload (after volume administration with or without excretion dysfunction) An acute event can also be caused by sepsis. Examples include acute coronary syndrome (i.e. the various forms of UAP and the various forms of MI defined elsewhere in this application).

Furthermore, patients presenting with symptoms of acute decompensation who suffer from a chronic disease but are presently suffering from an additional acute cardiac event (above-mentioned patient group 2) show a non-increased or only weakly increased level of NT-proBNP but a highly increased level of NT-proANP at the time of admission to a hospital. Furthermore, in these patients the level of NT-proANP drops off rapidly within the first 12 hours after admission. This pattern is quite similar to the pattern observed in the patients who are not suffering from a chronic cardiac disease (patient group 1).

Furthermore, patients presenting with symptoms of acute decompensation caused by a chronic cardiac disease (above-mentioned patient group 3) show highly increased values of NT-proBNP and NT-proANP already at the time of admission to a hospital. The time-course of the levels of both biomarkers runs approximately parallel for the first 24 hours. Notably, the value of NT-proBNP is already highly increased at the time of admission. Furthermore, in the case of a chronic cardiac disease the levels of NT-proANP and NT-proBNP do not change as much over the first day after admission as in the case of an acute cardiac event.

Also, the measured level of NT-proANP can depend on the severity of the cardiac disease: The more severe the cardiac disease, the higher the level of NT-proANP. Furthermore, the end of the acute event can be associated with a decrease of NT-proANP. In contrast, NT-proBNP reflects (mainly) the chronic cardiac disease and shows only slow changes. The measured levels may not only indicate presence or absence of a cardiac disease (particularly an acute cardiac event or a chronic cardiac disease) but also extent or severity of the disease. The measured levels therefore reflect the clinical continuum between patients suffering from a minor or a more severe cardiac disease. E.g., a very high level of NT-proANP indicates the presence of a more severe acute cardiac event.

The levels of NT-proANP and NT-proBNP in patients suffering from a chronic disease but presently suffering from an additional acute cardiac event (above-mentioned patient group 2) are similar to the levels in patient group 1 (acute cardiac event, but no chronic disease). Thus, the present invention appears to allow a diagnosis of an acute cardiac event independently of whether the patient has previously suffered from a chronic cardiac disease or not.

According to the present invention, the term “non-increased level of NT-proBNP” preferably corresponds to a plasma level of NT-proBNP of less than 125 pg/ml, particularly of less than 76 pg/ml, more particularly of less than 50 pg/ml.

According to the present invention, the term “weakly increased level of NT-proBNP” preferably corresponds to a plasma level of NT-proBNP of 125 to 1000 pg/ml, particularly 125 to 900 pg/ml, more particularly 125 to 750 pg/ml.

According to the present invention, the term “highly increased level of NT-proBNP” preferably corresponds to a plasma level of NT-proBNP of more than 3000 pg/ml.

According to the present invention, the term “increased level of NT-proANP” preferably corresponds to a plasma level of NT-proANP of more than 3000 pg/ml, more particularly of more than 4000 pg/ml, even more particularly of more than 7000 pg/ml, and most particularly of more than 10000 pg/ml. Further preferred level(s) can be derived from the upper limits of the reference intervals as described in Example 2.

It is evident that the combined information from NT-proANP and NT-proBNP may also be expressed differently. E.g. the relationship between NT-proANP and NT-proBNP may also be expressed as a “ratio”. In general, the higher the measured ratio of NT-proANP to NT-proBNP in a sample of a patient showing symptoms of acute cardiac decompensation is, the more likely is it that the patient is suffering from an acute cardiac event (and belongs to above mentioned patient group 1 or 2). Particularly, a ratio of more than 35, more particularly a ratio of more than 50, most particularly a ratio of more than 70, indicates the presence of an acute cardiac event.

This aspect of the present invention is a subject-matter of WO 2006/131529.

In a further embodiment of the present subject-matter of the invention, the amounts of NT-proANP and NT-proBNP can be used for the diagnosis of myocardial dysfunction. To this end, preferably the ratio of NT-proANP to NT-proBNP is used.

Another indicator of heart failure, particularly systolic dysfunction, is the “left ventricular ejection fraction” (LVEF) which is also known as “ejection fraction”. People with a healthy heart usually have an unimpaired LVEF, which is generally described as above 50%. Most people with a systolic dysfunction which is symptomatic generally have an LVEF of 40% or less.

Particularly, the present aspect of the invention relates to the diagnosis of diastolic dysfunction. More particularly, the aspect relates to distinguishing a diastolic from a systolic dysfunction. The term “diastolic dysfunction” is known to the person skilled in the art. In diastolic dysfunction, the ejection fraction is normal and the end-diastolic pressure is elevated; there is diminished capacity to fill at low left-atrial pressures. In contrast, in “systolic dysfunction” the LVEF is reduced and the end-diastolic pressure is normal.

Certain patients may show a mixed form of diastolic and systolic dysfunction. For example, a severe diastolic dysfunction may lead to a systolic dysfunction and the character of the dysfunction under this borderline condition may be mixed. It is evident to the person skilled in the art, that such a mixed form of diastolic and systolic dysfunction will most likely be present at the border values between the ratios (of ANP-type to BNP-type peptide) indicative of diastolic and systolic dysfunction, e.g. in a range of 3.5 to 7 (pg/ml of NT-proANP to pg/ml of NT-proBNP).

Furthermore, the ratio of ANP-type to BNP-type peptide is “inversely correlated” with the severity of the diastolic dysfunction. This means that the lower the ratio, the more severe is the diastolic dysfunction and vice versa. However, as evident from the context of the specification, a very low ratio (e.g. below 4.5 pg/ml of NT-proANP to pg/ml of NT-proBNP) indicates that the dysfunction is systolic or primarily systolic and a very high ratio indicates that no cardiac dysfunction is present.

A diastolic dysfunction may result in “diastolic heart failure”. A criterion for “diastolic heart failure” is the presence of a normal LVEF (above 50%) within three days after an episode of heart failure. Preferably objective evidence of diastolic dysfunction is also present (see above, e.g. abnormal left ventricular relaxation, filling or distensibility). Diagnosis of diastolic heart failure may also be made clinically, if there is reliable evidence of congestive heart failure and a normal LVEF, and that objective evidence of diastolic dysfunction obtained in the catheterization laboratory merely confirms diagnosis. This conclusion is consonant with the American College of Cardiology and the American Heart Association guidelines.

The principal difference between systolic and diastolic heart failure is the inability to relax or fill normally (diastolic heart failure) and the inability of the ventricle to contract normally and expel sufficient blood (systolic heart failure). Impaired relaxation or filling of the ventricle leads to an elevation of ventricular diastolic pressure at any given diastolic volume. Failure of relaxation can be functional, and transient, as during ischemia, or it can be chronicle, e.g. due to a stiffened, thickened ventricle.

According to the present invention, the term “diastolic heart failure” does not encompass conditions such as acute severe mitral regurgitation and other circulatory congestive states (e.g. congestive heart failure), which may also result in heart failure with normal ejection fraction. In these cases one would typically expect a relatively low ratio of ANP-type peptide to BNP-type peptide, e.g. a ratio of less than 5 pg/ml of NT-proAN P to pg/ml of NT-proBNP.

The information provided by the ratio NT-proANP to NT-proBNP may also serve for distinguishing cardiac dysfunctions in which one or both atria are affected from cardiac dysfunctions in which the one or both ventricles are affected. This may also relate to distinguishing the primary character of such dysfunctions, i.e. distinction of an atrial from a ventricular dysfunction. A higher ratio of ANP-type peptide to BNP-type peptide will indicate that the atrium is affected, whereas a lower ratio will indicate that the ventricle is affected. In more general terms, the ratio allows to distinguish whether the dysfunction is primarily atrial or primarily ventricular.

The combined information from ANP-type and BNP-type peptide ratio may also be expressed differently, e.g. as the ratio of the level of the BNP-type peptide to the ANP-type peptide. Any concentrations (molar or by weight) can be calculated easily.

Examples for known levels or ratios are given below. For example, a ratio of the plasma levels of less than 20, preferably of less than 17, (pg/ml of NT-proANP to pg/ml of NT-proBNP) indicates the presence of a cardiac dysfunction. In another example, a ratio of the plasma levels of more than 20, preferably more than 23, (pg/ml of NT-proANP to pg/ml of NT-proBNP) indicates the absence of a cardiac dysfunction.

Furthermore, a ratio of the plasma levels in the range of 6 to 20, preferably of 7 to 17, (pg/ml of NT-proANP to pg/ml of NT-proBNP) indicates the presence of a diastolic dysfunction. A ratio in the range of 15 to 20 (pg/ml of NT-proANP to pg/ml of NT-proBNP) indicates the presence of a less severe diastolic dysfunction. A ratio in the range of 6 to 15 (pg/ml of NT-proANP to pg/ml of NT-proBNP) indicates the presence of a more severe diastolic dysfunction. A ratio of less than 6, preferably less than 4.5, indicates the presence of a systolic dysfunction.

For example, a plasma level in the range of 125 to 700 pg/ml of NT-proBNP may indicate the presence of a diastolic dysfunction. A plasma level in the range of 125 to 250 pg/ml of NT-proBNP may indicate the presence of a less severe diastolic dysfunction. A plasma level in the range of 250 to 700 pg/ml of NT-proBNP may indicate the presence of a more severe diastolic dysfunction. A plasma level of more than 700 pg/ml, preferably of more than 1000 pg/ml of NT-proBNP may indicate the presence of a primarily systolic dysfunction. At a level of less than 125 pg/ml, preferably of less than 80 pg/ml, the presence of a diastolic dysfunction is unlikely.

This aspect of the present invention is a subject-matter of WO 2006/087373.

Furthermore, cardiac dysrhythmias may cause a rise in both NT-proANP and NT-proBNP and, in consequence, a high ratio NT-proANP/NT-proBNP.

Also, in accordance with the present invention, further information about the pathophysiological state or, respectively, a change thereof can be obtained from the amount of GDF-15. With respect to the reference values cited beforehand, an increased amount of GDF-15 is indicative for inflammatory processes which may occur in the myocard, whereas with respect to the reference values, a decreased amount of GDF-15 is indicative for the absence of inflammatory processes in the myocard. Thus, in a preferred embodiment of the method of the present invention, an increased amount of GDF-15 is indicative for inflammatory processes which may occur in the myocard, whereas a decreased amount of GDF-15 is indicative for the absence of inflammatory. As GDF-15 is a general marker for inflammatory processes and not specific for the myocard, further information gained from the amounts of the other markers measured in the method of the present invention is necessary to ensure that inflammatory processes are going on in the myocard.

In particular, when the amounts of GDF-15 are elevated in combination with elevated amounts of NT-proBNP and/or NT-proANP, this is indicative for inflammatory processes in the myocard.

Reference values for GDF-15 referred to herein: preferably 600 pg/ml, more preferably 800 pg/ml, even more preferably 1200 pg/ml, most preferably 1800 pg/ml.

An amount of GDF-15 equal to or higher than the values cited beforehand are indicative for an inflammation process (which may occur in the myocard)

Patients suffering from myocardial infarction MI can be diagnosed using cardiac troponins, preferably troponin T or I, most preferably troponin T. Myocardial infarction is regarded as being caused by a necrotic state of the myocard, i.e. cell death. Cardiac troponins are released following cell death and can hence be used for the diagnosis of MI. If the amount of Troponin T in the blood is elevated, i.e. above 0.1 ng/ml, an acute cardiovascular event, in particular myocardial infarction MI, is assumed and the patient is treated accordingly. However, it is known that cardiac troponins are also be released (in small amounts) in pathophysiological states preceding cell death, e.g. ischemia. Preferably, the amount of a cardiac troponin, particularly troponin, is determined with a very sensitive troponin t test system in order to allow a reliable determination of very low cardiac troponin amounts, preferably said test system is capable of determining amounts of 0.002 ng/ml troponin in a sample, preferably, in a blood, blood serum or blood plasma sample. A particularly preferred Troponin T assay in the context of the present invention is the Elecsys® 2010 analyzer (Roche Diagnostics) with a detection limit of from 0.001 ng/ml to 0.0015 ng/ml, in general 0.0015 ng/ml.

Therefore, in accordance with the present invention, and with respect to the healthy reference values cited beforehand, an amount of cardiac troponin, in particular troponin T equal to or higher (elevated) than the healthy reference value is indicative for myocardial ischemia and hypoxia and/or necrosis, whereas with respect to the reference values, a decreased amount of cardiac troponin, in particular troponin T is indicative for the absence of myocardial ischemia and hypoxia and/or necrosis. Thus, in a preferred embodiment of the method of the present invention, an elevated amount of cardiac troponin, in particular troponin T is indicative for myocardial ischemia and hypoxia and/or necrosis.

In the context of the present invention, values for the cardiac troponin equal to or higher than the following reference values are considered indicative for the presence of hypoxia, ischemia, and/or necrosis.

Cardiac troponin, preferably troponin I or troponin T, in particular troponin T referred to herein:

in case of hypoxia/necrosis: preferably 0.002 pg/ml, more preferably 0.004 pg/ml, most preferably 0.006 pg/ml.
in case of necrosis: 0.1 pg/ml

In case the amounts of a cardiac troponin and a natriuretic peptide (NT-proBNP, NT-proANP) are both elevated, this is indicative of an acute cardiovascular event. If in this embodiment the level of NT-proANP is elevated, this is indicative that the event has occurred recently.

Accordingly, the method of the present invention provides for a highly reliable monitoring result. The techniques which are currently used to resolve this issue are time consuming and cost intensive. The method of the present invention, however, allows a reliable, fast and less cost-intensive diagnosis and can be implemented even in portable assays, such as test stripes. Therefore, the method is particularly well suited for monitoring heart failure patients. Thanks to the findings of the present invention, a suitable therapy for a subject can be reliably selected. Severe side effects caused by not initiating a treatment or a wrong treatment of patients can be avoided.

In particular, the following changes in the levels of the various peptides determined in accordance with the present invention are characteristic of the following pathological states or changes thereof:

TABLE I
Monitoring of clinically stable or unstable cardiac patients
Comparison to previous result

| | NT-pro BNP | NT-pro ANP | TNT | GDF 15 | preferred Interpretation |
|---|---|---|---|---|---|
| 1 | unchanged | unchanged | unchanged | unchanged | stable patient |
| 2 | increased | increased | increased | increased | progressive disease |
| 3 | in- or decreased | in- or decreased | unchanged | unchanged | functional change in rhythm |
| 4 | unchanged | in- or decreased | unchanged | unchanged | functional acute event |
| 5 | unchanged | unchanged | increased | unchanged | necrotic event |
| 6 | increased | in- or decreased | increased | unchanged | necrotic event with functional change |
| 7 | unchanged | unchanged | unchanged | in- or decreased | inflammatory process not cardiac |
| | | | | | related |
| 8 | decreased | decreased | decreased | decreased | improvement |

The present invention also relates to a method of diagnosing and/or deciding which therapy/medication is to be applied in an apparently stable subject suffering from heart failure and undergoing a change in its pathophysiological state, said method comprising
a) repeatedly determining, within given time intervals, the amounts of the following peptides:
NT-proANP or a variant thereof;
NT-proBNP or a variant thereof;
a cardiac troponin or a variant thereof;
GDF-15 or a variant thereof; and
in a sample of the said subject; and
b) comparing the amounts measured in each determination of each of the markers cited in step a) and comparing these with reference amounts; and
c) diagnosing and/or deciding, in accordance with the amounts determined in a) and/or the information obtained in b), which medication is to be applied to the subject.

The term “diagnosing” as used herein means assessing as to whether a certain medication should be administered to a subject having undergone the test according to the present invention. The medication is selected from the following:

In the context of the present invention, in particular in connection with the Table I, a deviation of ≧20%, preferably ≧40%, in particular ≧60% from the reference value are considered to be an increase or decrease which is regarded to be significant (i.e. showing that the pathophysiological state has changed).

### A)

agents effecting cardiac function, preferably: beta blockers like proprenolol, metoprolol, bisoprolol, carvedilol, bucindolol, nebivolol; nitrates; adrenergic agonists, like dobutamine, dopamine, epinephrine, isoprotenerol, norepinephrine, phenylephrine; positive inotropic agents, like digoxin, digitoxin; diuretics, in particular loop diuretics, thiazide and thiazide-like diuretics, K-sparing diuretics, type I mineralocorticoid receptor antagonists, carbonic anhydrase inhibitors, vasopressure antagonists.

The information whether these agents should be administered is provided if an elevated level of a natriuretic peptide is measured. Suitable natriuretic peptides are BNP, NT-proBNP, ANP, NT-proANP; preferably BNP or NT-proBNP, in particular NT-proBNP.

When a level of natriuretic peptide of, in the case of NT-proBNP, >300 pg/ml, preferably >500 pg/ml, more preferably >800 pg/ml, still more preferably >2000 pg/ml is reached, one or more of the above-cited drugs should be administered.

### B)

anti-inflammatory drugs, preferably: ACE inhibitors, in particular Enalapril, Captopril, Ramipril, Trandolapril; angiotensin receptor antagonists and aldosterone antagonists, in particular Losartan, Valsartan, Irbesartan, Candesartan, Telmisartan, Eprosartan, Spironolactone; statines, in particular Atorvastatin, Fluvastatin, Lovastatin, Pravastatin, Rosuvastatin, Simvastatin; NSAIDS; selective COX-2 inhibitors

The information whether these agents should be administered is provided if an elevated level of GDF-15 which is indicative for inflammatory processes is measured.

When a level of GDF-15 of >800 pg/ml, preferably >1200 pg/ml, more preferably >1500 pg/ml, in particular >2000 pg/ml is reached, one or more of the above-cited drugs should be administered.

### C)

In general, Troponin I and/or T, in particular Troponin T, is indicative of an existing myocardial necrosis and the extent of the necrosis; in case no drop in the level of Troponin T/I is observed, then this peptide indicates heart failure and/or vascular stenosis which can be treated by percutane coronary intervention.

The information whether these agents should be administered if an elevated level of Troponin I and/or Troponin T, in particular Troponin T which is indicative for heart failure or vascular stenosis is measured.

The present invention further encompasses a device for monitoring an apparently stable subject suffering from heart failure comprising:
a) means for repeatedly determining the amounts of the following peptides:
NT-proANP or a variant thereof;
NT-proBNP or a variant thereof;
a cardiac troponin or a variant thereof;
GDF-15 or a variant thereof;
in a sample of the said subject; and
means for comparing the amounts measured in each determination of each of the markers cited in step a) and for comparing these with reference amounts;
whereby it is assessed, based on the differences in the determined amounts in one or more of the above-cited markers, whether the subject is stable or has undergone a change in its physiological state;
whereby the device is adapted for carrying out the method of the present invention referred to above.

The term “device” as used herein relates to a system of means comprising at least the aforementioned means operatively linked to each other as to allow the prediction. Preferred means for determining the amount of a one of the aforementioned polypeptides as well as means for carrying out the comparison are disclosed above in connection with the method of the invention. How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. The computer unit, preferably, comprises a database including the stored reference amounts or values thereof recited elsewhere in this specification as well as a computer-implemented algorithm for carrying out a comparison of the determined amounts for the polypeptides with the stored reference amounts of the database. Computer-implemented as used herein refers to a computer-readable program code tangibly included into the computer unit. Alternatively, where means such as test stripes are used for determining the amount of the peptides or polypeptides, the means for comparison may comprise control stripes or tables allocating the determined amount to a reference amount. The test stripes are, preferably, coupled to a ligand which specifically binds to the peptides or polypeptides referred to herein. The strip or device, preferably, comprises means for detection of the binding of said peptides or polypeptides to the said ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test stripes or electronic devices which merely require loading with a sample. The results may be given as output of raw data which need interpretation by the clinician. Preferably, the output of the device is, however, processed, i.e. evaluated, raw data, the interpretation of which does not require a clinician. Further preferred devices comprise the analyzing units/devices (e.g., biosensors, arrays, solid supports coupled to ligands specifically recognizing the natriuretic peptide, Plasmon surface resonance devices, NMR spectrometers, mass-spectrometers etc.) and/or evaluation units/devices referred to above in accordance with the method of the invention.

Also, the present invention relates to a device for diagnosing and/or deciding which therapy/medication is to be applied in an apparently stable subject suffering from heart failure and undergoing a change in its pathophysiological state, comprising:
a) means for repeatedly determining the amounts of the following peptides:
NT-proANP or a variant thereof;
NT-proBNP or a variant thereof;
a cardiac troponin or a variant thereof;
GDF-15 or a variant thereof;
in a sample of the said subject; and
b) means for comparing the amounts measured in each determination of each of the markers cited in step a) and for comparing these with reference amounts;
whereby, based on the differences in the determined amounts in one or more of the above-cited markers, a diagnosis/decision is made on the therapy/medication,
whereby the device is adapted for carrying out the method of the present invention'referred to above.

Moreover, the present invention relates to a kit adapted for carrying out the methods of the present invention referred to above comprising:
a) means for repeatedly determining the amounts of the following peptides:
NT-proANP or a variant thereof;
NT-proBNP or a variant thereof;
a cardiac troponin or a variant thereof;
GDF-15 or a variant thereof;
in a sample of an apparently stable subject suffering from heart failure; and
b) means for comparing the amounts measured in each determination of each of the markers cited in step a) and for comparing these with reference amounts;
whereby, based on the differences in the determined amounts in one or more of the above-cited markers, the methods of the present invention are carried out,
whereby the kit is adapted for carrying out the method of the present invention referred to above. Preferably, the kit comprises instructions for carrying out the said method of the present invention.

The term “kit” as used herein refers to a collection of the aforementioned means, preferably, provided in separately or within a single container. The container, also preferably, comprises instructions for carrying out the method of the present invention.

All references cited in this specification are herewith incorporated by reference with respect to their entire disclosure content and the disclosure content specifically mentioned in this specification.

The following examples shall merely illustrate the invention. They shall not be construed, whatsoever, to limit the scope of the invention.

### Example 1

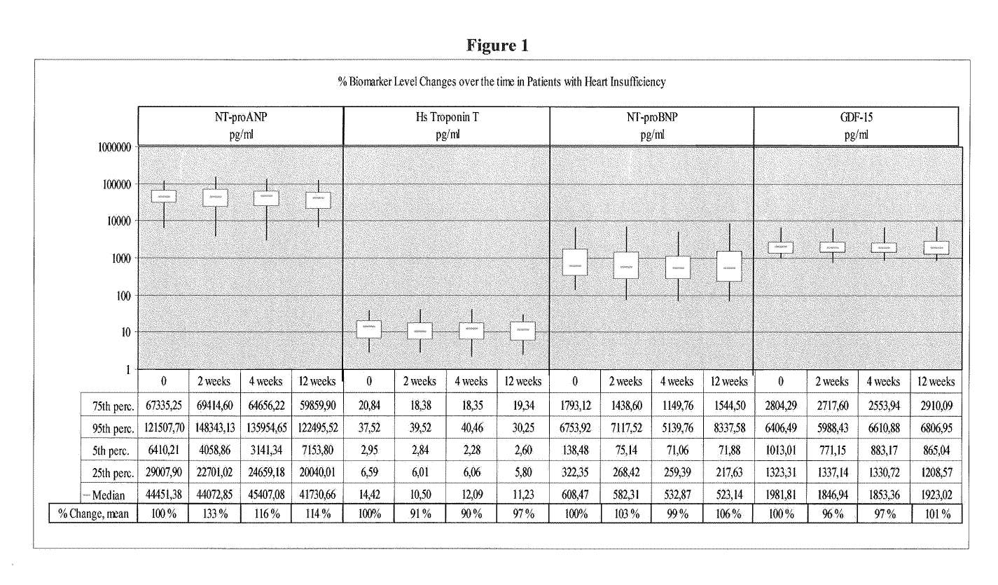

In a total of 41 apparently stable patients with heart failure, the levels of NT-proBNP, NT-proANP, hs troponin T and GDF 15 were determined, after 2 weeks, 4 weeks and 12 weeks. The study was continued over a period of in total 3 months. The median of the amounts did not change (see FIG. 1). However, patients could be identified showing stable amounts of all markers over the entire time period, as well as patients which showed a significant change or in one or two of the markers or even in all markers.

FIG. 1 depicts the variation in the markers mentioned above, depending on the 5th, 25th, 75th and 95th percentile.

Table 1 shows a plot of the respective values and the standard deviation.

TABLE 1
% Biomarker Level Changes over the time in Patients with Heart Insufficiency

| | 0 | 2 weeks | 4 weeks | 12 weeks | 0 | 2 weeks | 4 weeks | 12 weeks |
|---|---|---|---|---|---|---|---|---|
| | NT-proANP pg/ml | | | | Hs Troponin T pg/ml | | | |
| 75th perc. | 67335.25 | 69414.60 | 64656.22 | 59859.90 | 20.84 | 18.38 | 18.35 | 19.34 |
| 95th perc. | 121507.70 | 148343.13 | 135954.65 | 122495.52 | 37.52 | 39.52 | 40.46 | 30.25 |
| 5th perc. | 6410.21 | 4058.86 | 3141.34 | 7153.80 | 2.95 | 2.84 | 2.28 | 2.60 |
| 25th perc. | 29007.90 | 22701.02 | 24659.18 | 20040.01 | 6.59 | 6.01 | 6.06 | 5.80 |
| Median | 44451.38 | 44072.85 | 45407.08 | 41730.66 | 14.42 | 10.50 | 12.09 | 11.23 |
| % Change, | 100% | 133% | 116% | 114% | 100% | 91% | 90% | 97% |
| mean | | | | | | | | |
| | NT-proBNP pg/ml | | | | GDF-15 pg/ml | | | |
| 75th perc. | 1793.12 | 1438.60 | 1149.76 | 1544.50 | 2804.29 | 2717.60 | 2553.94 | 2910.09 |
| 95th perc. | 6753.92 | 7117.52 | 5139.76 | 8337.58 | 6406.49 | 5988.43 | 6610.88 | 6806.95 |
| 5th perc. | 138.48 | 75.14 | 71.06 | 71.88 | 1013.01 | 771.15 | 883.17 | 865.04 |
| 25th perc. | 322.35 | 268.42 | 259.39 | 217.63 | 1323.31 | 1337.14 | 1330.72 | 1208.57 |
| Median | 608.47 | 582.31 | 532.87 | 523.14 | 1981.81 | 1846.94 | 1853.36 | 1923.02 |
| % Change, | 100% | 103% | 99% | 106% | 100% | 96% | 97% | 101% |
| mean | | | | | | | | |

### Example 2

A total of 39 patients suffering from atrial fibrillation was treated with electrocardioversion. After that, 29 patients returned to cardiac sinus rhythm, 10 patients continued with cardiac fibrillation. The levels of NT-proBNP, NT-proANP, hs troponin T and GDF 15 were determined, before and immediately after the electrocardioversion and 11 days later. It was shown that the change in the cardiac rhythm impacts the amounts of the natriuretic peptides, but not of GDF-15 and of troponin T.

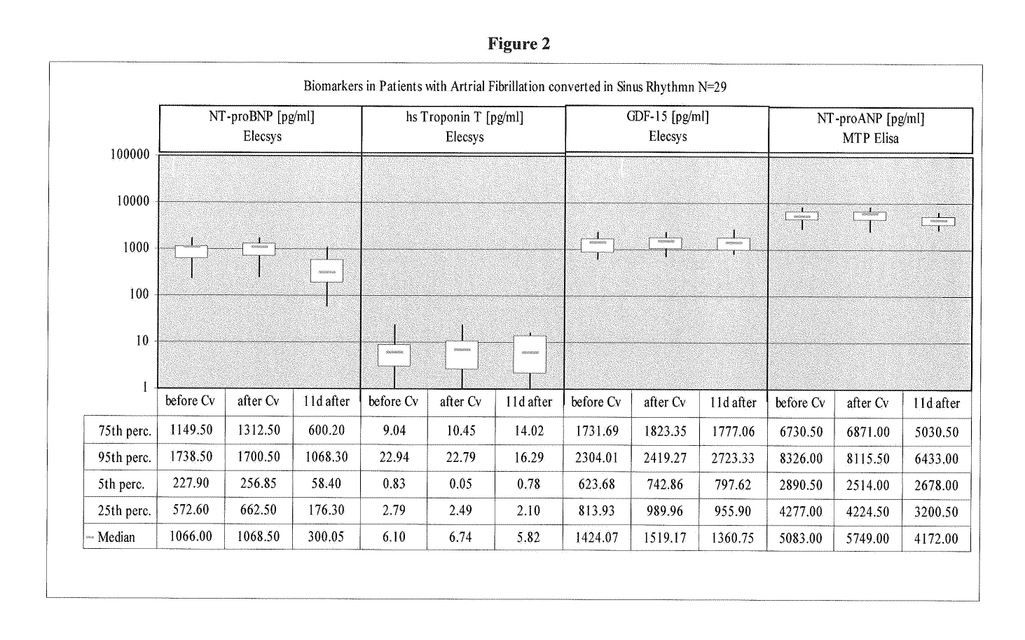

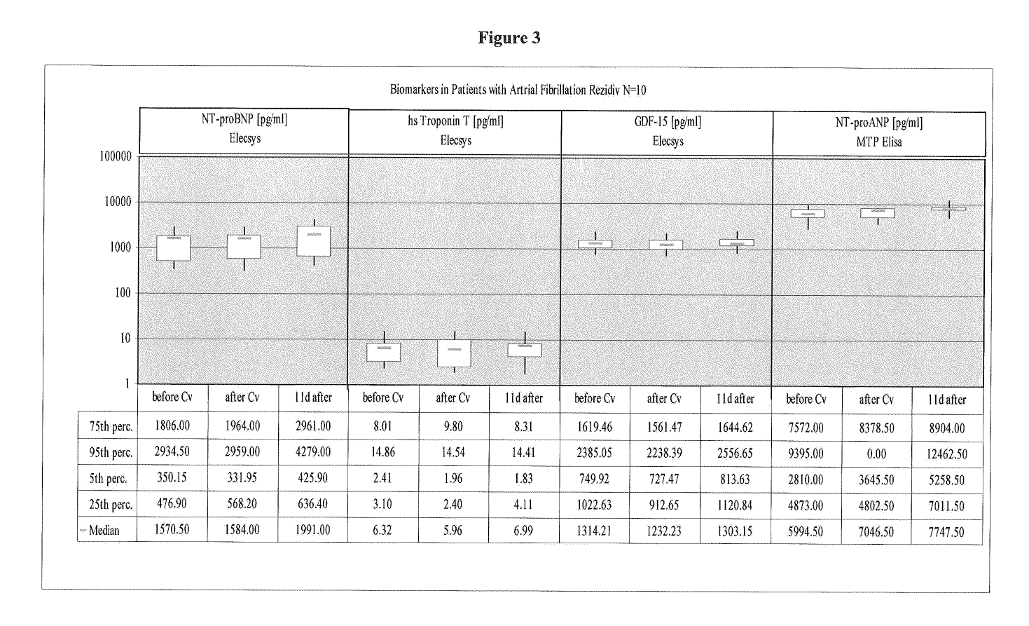

FIGS. 2 and 3 show the levels of the respective peptides, depending on the time interval and the pathophysiological state of the patient.

TABLE 2
Biomarkers in Patients with Atrial Fibrillation converted in Sinus Rhythm N = 29

| | before Cv | after Cv | 11 d after | before Cv | after Cv | 11 d after |
|---|---|---|---|---|---|---|
| | NT-proBNP [pg/ml] Elecsys | | | hs Troponin T [pg/ml] Elecsys | | |
| Median | 1066.00 | 1068.50 | 300.05 | 6.10 | 6.74 | 5.82 |
| 75th perc. | 1149.50 | 1312.50 | 600.20 | 9.04 | 10.45 | 14.02 |
| 95th perc. | 1738.50 | 1700.50 | 1068.30 | 22.94 | 22.79 | 16.29 |
| 5th perc. | 227.90 | 256.85 | 58.40 | 0.83 | 0.05 | 0.78 |
| 25th perc. | 572.60 | 662.50 | 176.30 | 2.79 | 2.49 | 2.10 |
| | GDF-15 [pg/ml] Elecsys | | | NT-proANP [pg/ml] MTP Elisa | | |
| Median | 1424.07 | 1519.17 | 1360.75 | 5083.00 | 5749.00 | 4172.00 |
| 75th perc. | 1731.69 | 1823.35 | 1777.06 | 6730.50 | 6871.00 | 5030.50 |
| 95th perc. | 2304.01 | 2419.27 | 2723.33 | 8326.00 | 8115.50 | 6433.00 |
| 5th perc. | 623.68 | 742.86 | 797.62 | 2890.50 | 2514.00 | 2678.00 |
| 25th perc. | 813.93 | 989.96 | 955.90 | 4277.00 | 4224.50 | 3200.50 |

TABLE 3
Biomarkers in Patients with Artrial Fibrillation, recurrence of atrial fibrillation N = 10

| | before Cv | after Cv | 11 d after | before Cv | after Cv | 11 d after |
|---|---|---|---|---|---|---|
| | NT-proBNP [pg/ml] Elecsys | | | hs Troponin T [pg/ml] Elecsys | | |
| Median | 1570.50 | 1584.00 | 1991.00 | 6.32 | 5.96 | 6.99 |
| 75th perc. | 1806.00 | 1964.00 | 2961.00 | 8.01 | 9.80 | 8.31 |
| 95th perc. | 2934.50 | 2959.00 | 4279.00 | 14.86 | 14.54 | 14.41 |
| 5th perc. | 350.15 | 331.95 | 425.90 | 2.41 | 1.96 | 1.83 |
| 25th perc. | 476.90 | 568.20 | 636.40 | 3.10 | 2.40 | 4.11 |
| | GDF-15 [pg/ml] Elecsys | | | NT-proANP [pg/ml] MTP Elisa | | |
| Median | 1314.21 | 1232.23 | 1303.15 | 5994.50 | 7046.50 | 7747.50 |
| 75th perc. | 1619.46 | 1561.47 | 1644.62 | 7572.00 | 8378.50 | 8904.00 |
| 95th perc. | 2385.05 | 2238.39 | 2556.65 | 9395.00 | 0 | 12462.50 |
| 5th perc. | 749.92 | 727.47 | 813.63 | 2810.00 | 3645.50 | 5258.50 |
| 25th perc. | 1022.63 | 912.65 | 1120.84 | 4873.00 | 4802.50 | 7011.50 |

What is claimed is:

1. A method for monitoring an apparently stable subject suffering from heart failure, the method comprising:
repeatedly determining, within a given time interval, an amount of each of the peptide markers N-terminal pro-atrial natriuretic peptide (NT-proANP), N-terminal pro-brain natriuretic peptide (NT-proBNP), a cardiac troponin, and growth differentiation factor 15 (GDF-15) in a sample from the subject,
comparing the amounts determined with reference amounts of the respective markers, and
assessing, based on the differences in the determined amounts in one or more of the markers, whether the subject is stable or has undergone a change in pathophysiological state.

2. The method of claim 1, wherein the cardiac troponin is troponin I or troponin T.

3. The method of claim 1, wherein an increase or decrease of >20% relative to the reference amount is indicative of a change in pathophysiological state of the individual.

4. The method of claim 3, wherein the increase or decrease is >40%.

5. The method of claim 1, wherein the reference values are developed from a healthy individual, and a value equal to or greater than the reference amount is indicative of a deterioration in the pathophysiological state.

6. The method of claim 5, wherein the reference value for troponin T is 0.002 pg/ml and the reference value for GDF-15 is 600 pg/ml.

7. The method of claim 1, wherein the peptides are NT-proBNP and NT-proANP and the reference values are developed from an individual suffering from heart failure.

8. The method of claim 7, wherein the reference value for NT-proBNP is 125 pg/ml and the reference value for NT-proANP is 800 pg/ml.

9. The method of claim 7, wherein a deviation from the reference value is indicative of an amelioration or a deterioration of the pathophysiological state of the individual.

10. The method of claim 1, wherein the following pathophysiological states of the individual can be determined: stable heart failure; progressive disease; functional change in rhythm; functional acute event; necrotic event; necrotic event with functional change; inflammatory process not cardiac related; improvement of heart failure.

11. The method of diagnosing and/or deciding which therapy/medication is to be applied in an apparently stable subject suffering from heart failure and undergoing a change in physiological state, the method comprising
repeatedly determining, within a given time interval, an amount of the peptide markers NT-proANP, NT-proBNP, a cardiac troponin, and GDF-15 in a sample from the subject,
comparing the amounts measured in each determination of each of the markers and comparing these with a reference amount for each marker, and
diagnosing and/or deciding, in accordance with the amounts determined, which therapy/medication is to be applied to the subject.

12. The method according to claim 11, wherein the medication is selected from the group consisting of beta blockers, nitrates, adrenergic agonists, positive inotropic agents, diuretics, angiotensin receptor antagonists, aldosterone antagonists, NSAIDS, selective Cox-2 inhibitors, statins, ACE inhibitors, and percutane coronary intervention.

13. A device for monitoring an apparently stable subject suffering from heart failure comprising:
means for repeatedly determining, in a given time interval, amounts of NT-proANP, NT-proBNP, a cardiac troponin, and GDF-15 peptide markers, and
means for comparing the amounts determined with reference amounts, whereby it is diagnosed, based on the differences in the determined amount in one or more of the above-cited markers, whether the subject is stable or has undergone a change in its physiological state
whereby the device is adapted for carrying out the method of the present invention as laid out in claim 1.

14. A device for diagnosing and/or deciding which medication is to be applied in a subject suffering from heart failure comprising:
means for repeatedly determining, in a given time interval, amounts of NT-proANP, NT-proBNP, a cardiac troponin, and GDF-15 peptide markers, and
means for comparing the amounts determined with reference amounts, whereby it is to be diagnosed, based on the differences in the determined amount in one or more of the above-cited markers, whether the subject is stable or has undergone a change in its physiological state
whereby the device is adapted for carrying out the method of the present invention as laid out in claim 1.

15. A kit adapted for carrying out the method of claim 1, the kit comprising:
means for repeatedly determining, in a given time interval, amounts of NT-proANP, NT-proBNP, a cardiac troponin, and GDF-15 peptide markers, and
instructions for carrying out the method, including comparing the amounts determined with reference amounts, whereby it is to be diagnosed, based on the differences in the determined amount in one or more of the peptide markers, whether the subject is stable or has undergone a change in physiological state.
